(19) United States
(12) Patent Application Publication
(10) Pub. No.: US 2021/0252212 A1
(43) Pub. Date: Aug. 19, 2021

(54) AN OSMOTIC ACTUATOR FOR AN INJECTING DEVICE AND AN INJECTION DEVICE COMPRISING SUCH AN OSMOTIC ACTUATOR

(71) Applicant: Subcuject ApS, Hellebæk (DK)

(72) Inventors: Claus Schmidt MØLLER, Fredensborg (DK); Tue Kjærgaard TOFT, København N (DK); Thomas STEENBERG, Roskilde (DK); Tomas GUNDBERG, Viby Sjælland (DK)

(21) Appl. No.: 17/252,230

(22) PCT Filed: Jun. 12, 2019
(86) PCT No.: PCT/DK2019/050185

§ 371 Date: Dec. 14, 2020

(30) Foreign Application Priority Data
Jun. 18, 2018 (DK) PA201870415
Jul. 10, 2018 (DK) PA201800373
Oct. 3, 2018 (DK) PA201870653
Feb. 1, 2019 (DK) PA201900149

(51) Int. Cl. A61M 5/145; A61J 1/06; A61M 5/142; A61K 9/00

(52) U.S. Cl. CPC A61M 5/145; A61J 1/065; A61M 2005/14513; A61K 9/0004; A61M 5/14248

(57) ABSTRACT

An osmotic actuator (110) for an injection device (100) is disclosed, which osmotic actuator (110) comprises a pressure chamber (180; 280; 380) having one or more outlets (182; 282; 295; 395) and containing a draw solution, one or more osmotic membranes (130), a cavity (140) containing water, and a dilution-compensating arrangement, wherein the one or more osmotic membranes (130) forms a part of an internal surface area of the pressure chamber (180; 280; 380), wherein the cavity (140) containing water abuts at least part of an external surface of the one or more osmotic membranes (130), and wherein the dilution-compensating arrangement is arranged to compensate for the dilution of the draw solution near the one or more osmotic membranes (130), which occurs when water from the cavity (140) enters the pressure chamber (180; 280; 380) through the one or more osmotic membranes (130). Furthermore, an injection device (100) comprising such an osmotic actuator (110) is disclosed.

### FIELD OF THE INVENTION

The present invention relates to an osmotic actuator to be used in an injection device, and which is capable of providing a stabile flow rate during the use of the device.

### BACKGROUND OF THE INVENTION

Using an osmotic actuator as drive unit for an injection device is very attractive in situations, where the drug must be injected slowly into the patient, e.g. when a large volume of drug must be injected. An osmotic actuator is capable of providing a very high pressure, but the build-up time for the pressure can easily be controlled by the type and size of the osmotic membrane and by the concentration and type of the osmotic draw solution. The pressure and the increased volume in the actuator due to feed water passing through the osmotic membrane and into the actuator can be used for moving a plunger in a cartridge or to squeeze a flexible reservoir.

WO 2017/129191 describes several embodiments of a wearable injection device equipped with a drive unit in the form of an osmotic actuator. One internal side of a pressure chamber of the osmotic actuator containing a draw solution is formed by an osmotic membrane, which is in contact with a water reservoir on the outside. When water is drawn in through the osmotic membrane due to the osmotic process, a pressure builds up and the excess water is pressed out through an outlet and adapted to move the plunger in a cartridge.

However, when water enters the osmotic actuator through the osmotic membrane, the draw solution inside the osmotic actuator is diluted and a border layer of water near the osmotic membrane inside the pressure chamber reduces the osmotic potential and causes the flow rate to fall over time.

### BRIEF DESCRIPTION OF THE INVENTION

It is an objective of the present invention to provide an osmotic actuator for an injection device, which delivers a more constant flow rate during the time of use of the device than what is known from osmotic actuators in the prior art.

The present invention relates to an osmotic actuator for an injection device adapted for subcutaneous injection of a medicament into the tissue of a user, which osmotic actuator comprises a pressure chamber having one or more outlets and containing a draw solution, one or more osmotic membranes, a cavity containing a solvent, and a dilution-compensating arrangement, wherein the one or more osmotic membranes forms a part of an internal surface area of the pressure chamber, and wherein the cavity containing the solvent abuts at least part of an external surface of the one or more osmotic membranes so that at least one common boundary between the pressure chamber and the cavity containing the solvent is formed by the one or more osmotic membranes, and wherein the dilution-compensating arrangement is arranged to compensate for the dilution of the draw solution near the one or more osmotic membranes, which dilution occurs when solvent from the cavity enters the pressure chamber through the one or more osmotic membranes.

If this dilution is not compensated for, it will cause the flow through the one or more outlets of the pressure chamber to decrease as the osmotic gradient across the one or more osmotic membranes decreases.

In preferred embodiments of the invention, the solvent is water, preferably demineralized water.

In preferred embodiments of the invention, the osmotic actuator is arranged so that no additional draw solution or osmotic agent is supplied to the pressure chamber during the use of the osmotic actuator.

In a first embodiment of the invention, the dilution-compensating arrangement comprises a rotating element defining an axis of revolution and with an upper part having one or more protrusions spaced equally around the axis of revolution, the rotating element being positioned in the pressure chamber and arranged to rotate at least during a part of the time, in which the osmotic actuator is active.

Thereby the draw solution in the pressure chamber is circulated, the water entering the pressure chamber through the osmotic membrane is moved away from the osmotic membrane and a higher osmotic potential over the osmotic membrane is achieved.

In another embodiment of the invention, the rotating element has a lower part with three or more protrusions spaced around the axis of revolution, the lower part being arranged in an outlet flow of the pressure chamber in such a way that, when the draw solution flows towards the one or more outlets of the pressure chamber, the flow causes the rotating element to rotate around the axis of revolution.

Thereby no additional energy source for rotating the rotating element is needed.

In another embodiment of the invention, the axis of revolution of the rotating element is parallel to at least one of the one or more osmotic membranes.

This provides a better opportunity of creating a circulating flow in the actuator.

In another embodiment of the invention, the axis of revolution of the rotating element is perpendicular to at least one of the one or more osmotic membranes.

This allows the rotating element to be arranged on the widest measure of the osmotic actuator, whereby the diameter of the rotating element can be large and capable of transferring a high amount of mechanical energy to the draw solution.

In another embodiment of the invention, the rotating element is arranged to be in contact with the surface of at least one of the one or more osmotic membranes surface during rotation.

When the axis of revolution of the rotating element is perpendicular to a osmotic membrane and the rotating element is in contact with a surface thereof, the rotating element can scrape away the incoming water, thereby allowing draw solution with a high salinity to get to the osmotic membrane and increase the osmotic potential over the osmotic membrane.

In another embodiment of the invention, the dilution-compensating arrangement comprises a magnetic element being positioned outside the pressure chamber and arranged to apply a magnetic field within the pressure chamber.

This creates a diamagnetic effect within the pressure chamber that can be used for mixing purposes. As freshwater tends to be repelled by an external magnet stronger than salt water, a mixing effect is achieved in an inhomogeneous blend of freshwater and salt water.

In another embodiment of the invention, the draw solution contains magnetic particles having a positive or negative electrical surface charge.

Because the particles have a surface charge, they attract layers of positive and negative ions on the surface and bring them to the osmotic membrane by means of the magnetic field from the external magnet.

In another embodiment of the invention, the dilution-compensating arrangement comprises an anode and a cathode being connected through an electric resistance and positioned within the pressure chamber, wherein one of the anode and the cathode is positioned near the one or more osmotic membranes and the other is placed in an opposite side of the pressure chamber, so that the main part of the draw solution is positioned between the anode and the cathode.

Thereby, a current is produced in the draw solution and ions within the draw solution are transferred to the surface of the osmotic membrane, whereby a higher osmotic potential across the osmotic membrane is achieved.

In another embodiment of the invention, electric resistance is outside the pressure chamber and is in the form of an LED component.

Thereby the electric current induced can be used for providing a visual signal to the user, indicating that the osmotic actuator is active, and the injection device is in use.

In another embodiment of the invention, the electric resistance is outside the pressure chamber and is in the form of an LCD or e-ink display.

Thereby the electric current induced can be used for giving a message to the user about the functional status of the osmotic actuator and the injection device.

In another embodiment of the invention, the injection device further comprises a push-button for activating the injection device by pushing the push-button through a first distance whereby an electric connection between the anode and the cathode through the electric resistance is established, and wherein the push-button is arranged to move a second and shorter distance in the opposite direction at completion of an injection performed by the injection device whereby the electric connection between the anode and the cathode is broken.

By letting the current being disconnected at completion of the injection, a clear message can be given to the user that the injection has been fulfilled, e.g. by turning off a light signal, which was activated at activation of the injection device.

In another embodiment of the invention, the dilution-compensating arrangement comprises a division of the pressure chamber into a first compartment, into which the draw solution is released at activation of the injection device, and a second compartment arranged between the outlet and the first compartment, the second compartment being configured to be longer and narrower than the first compartment.

In this way, although both the first compartment and the second compartment form part of the pressure chamber and both have at least one boundary formed by an osmotic membrane, only a part of the total area of the one or more osmotic membranes is active from the beginning. During the injection and the use of the osmotic actuator, when dilution and concentration polarisation reduces the osmotic potential, a larger membrane area is taken into use as the salt passes into and through the second compartment, which in turn compensates for the lower osmotic potential and thereby smoothens out the flow rate over the full injection time. As a second advantage, this creates a higher flow in the second compartment due to a higher flow velocity and, thereby, a higher degree of mixing, which further optimises the osmotic potential.

In another embodiment of the invention, one or more protrusions are arranged in the second compartment to partly obstruct the flow through the second compartment.

Thereby the flow through the second compartment is more turbulent and, thereby, better mixed, and more salt water is guided to the osmotic membrane.

In another embodiment of the invention, the cross-sectional area of the second compartment varies along the length of the second compartment.

This gives the opportunity to configure the second compartment in a way, which results in a smooth and constant flow rate.

In another embodiment of the invention, the dilution-compensating arrangement comprises a pouch containing a salt solution, which pouch is positioned within the osmotic actuator and is punctured at activation of the injection device, whereby a slow outlet flow of salt solution from the pouch into the draw solution is obtained.

This ensures that fresh salt is added to the draw solution throughout the injection time so that a stabile degree of salination and, thereby, a stabile flow from the osmotic actuator is obtained.

In another embodiment of the invention, the dilution-compensating arrangement comprises a bluff body to be passed by incoming solvent in the osmotic actuator for creation of hydraulic vortices.

This creates some movement in the draw solution in the actuator and helps mix the draw solution.

In another embodiment of the invention, the dilution-compensating arrangement comprises chemicals added to the draw solution for increasing the speed of mixing by diffusion and/or for creating some movement in the draw solution.

In another embodiment of the invention, the dilution-compensating arrangement comprises a body, which is arranged within the osmotic actuator and to repeatedly shift position, thereby defining which one of a plurality of outlets is open at any given time, the other outlets being closed.

Such a shift between different outlets as well as the motion of the body within the pressure chamber help mixing the draw solution.

In another embodiment of the invention, the dilution-compensating arrangement comprises an expandable pressure chamber combined with a flow restrictor, which only allows a certain amount of fluid to pass through the one or more outlets.

This causes the pressure chamber to expand when the flow through the osmotic membrane is high and to relax and deliver the excess fluid through the outlet restrictor when the flow is lower, whereby a more smooth and constant flow is obtained.

In another embodiment of the invention, the draw solution within the pressure chamber is obtained by breaking a watertight barrier of a watertight salt depository arranged within the pressure chamber during activation of the osmotic actuator, whereby one or more salt tablets, crystal salt or an unsaturated, saturated or supersaturated salt solution initially contained by the watertight salt depository is brought into contact with water surrounding the watertight salt depository within the pressure chamber.

In this way a very simple supply of salt can be arranged inside the actuator, and a more complicated filling and emptying of a salt solution from a pouch is avoided.

In another embodiment of the invention, the watertight salt depository is a glass ampoule.

The advantage of using glass as barrier is that glass has outstanding barrier properties to fluids and that a glass ampoule can be easily broken by a crusher mechanism integrated in the osmotic actuator and actuated by the push-button.

In another embodiment of the invention, the breaking of the watertight barrier is fully or partly caused by a physical force applied to the watertight salt depository.

In another embodiment of the invention, a filling end of the glass ampoule is closed with a plug or a seal.

The advantage of using a plug or a kind of seal is that the filling percentage of the glass ampoule can be higher.

In another embodiment of the invention, the salt initially contained by the watertight salt depository is one or more of CaBr₂, CaCl₂, ZnBr₂, ZnCl₂, ZnI₂, LiBr, NH₂Cl or MgCl₂.

In another embodiment of the invention, the porosity of salt tablets or crystal salt initially contained by the watertight salt depository is reduced by stamping the salt crystals of the crystal salt before enclosing the crystal salt by the watertight barrier.

This can reduce the amount of air within the watertight salt depository to 15-25%, which is an advantage as air can cause the injection to be less smooth.

In another embodiment of the invention, the porosity of salt tablets or crystal salt initially contained by the watertight salt depository is reduced by further crystallization of salt in the voids between the salt crystals of the crystal salt.

This can bring the amount of air in the watertight salt depository further down.

In another embodiment of the invention, the porosity of salt tablets or crystal salt initially contained by the watertight salt depository is reduced by applying a vacuum to the crystal salt when enclosing the crystal salt by the watertight barrier.

This is an alternative way to reduce the amount of air, eliminating the extra process of crystalizing salt in the voids between the salt crystals.

In another embodiment of the invention, salt tablets or crystal salt initially contained by the watertight salt depository further comprise(s) an agent, which is reactive to or dissolvable in water.

This can accelerate the degradation of the salt and the start-up of the osmotic process.

In another embodiment of the invention, the initial amount of water surrounding the watertight salt depository within the pressure chamber is insufficient to dissolve the total amount of salt tablets or crystal salt initially contained by the watertight salt depository.

In this way, the dilution of the draw solution in the osmotic actuator during the osmotic process can be mitigated and a more stable flux can be obtained.

In an embodiment of the invention, the one or more osmotic membranes are flat sheet membranes.

In an aspect of the invention, it relates to an injection device comprising an osmotic actuator as described above.

In an embodiment of the invention, the injection device and the osmotic actuator are arranged so that the amount of medicament injected during the use of the injection device is within the range from 1 ml to 20 ml.

### FIGURES

In the following, some exemplary embodiments of the invention is described in further details with reference to the drawing, wherein

FIG. 1 is a perspective view of a wearable injection device according to an embodiment of the invention,

FIG. 2 is an exploded view of an osmotic actuator according to an embodiment of the invention,

FIG. 3 is a perspective view of a pressure chamber of an osmotic actuator according to an embodiment of the invention,

FIG. 4 is a perspective view of a rotating element of the pressure chamber shown in FIG. 3,

FIG. 5 is a top view of a pressure chamber shown in FIG. 3 without the rotating element,

FIG. 6 is a perspective view of a first alternative rotating element for an osmotic actuator according to an embodiment of the invention,

FIG. 7 is a perspective view of a second alternative rotating element for an osmotic actuator according to an embodiment of the invention,

FIG. 8 is a perspective view of a third alternative rotating element for an osmotic actuator according to an embodiment of the invention,

FIG. 9 is a sectional view of an outlet part of pressure chamber of an osmotic actuator according to an embodiment of the invention,

FIG. 10 is a top view of a pressure chamber according to another embodiment of the invention,

FIG. 11 is a perspective view of a pressure chamber according to yet another embodiment of the invention, and

FIG. 12 is a tip view of the pressure chamber shown in FIG. 11.

### DETAILED DESCRIPTION

Only parts necessary to understand the function of the different embodiments of the osmotic actuator 110 are included in the below description. The described ways of mixing and circulating the flow or to move ions/particles to the osmotic membrane 130 can be combined in numeral ways and are all within the scope of the invention. The terms “mixing” and “circulation” are used interchangeably in the description as means for making a more uniform draw solution within the pressure chamber 180, 280, 380 of the osmotic actuator 110.

The terms “up”, “down”, “upper”, “lower” and “downward” refer to the drawings and not necessarily to a situation of use.

The term “wearable injection device” 100 refers to a patient administrated injection device 100 for attachment to the body of a user and for subcutaneous injection of a medicament into the tissue of the user. The typical amount of medicament to be injected lies within the range from 1 ml to 20 ml. A wearable injection device 100 injects at a lower speed than, e.g., an auto injector and is often used when large amounts of drug must be injected. Thus, the amounts of liquid handled by the osmotic actuator of the present invention are orders of magnitude larger than the ones handled by so-called “micropumps”. Wearable injection devices 100 are normally for one-time use and are removed and discarded after use.

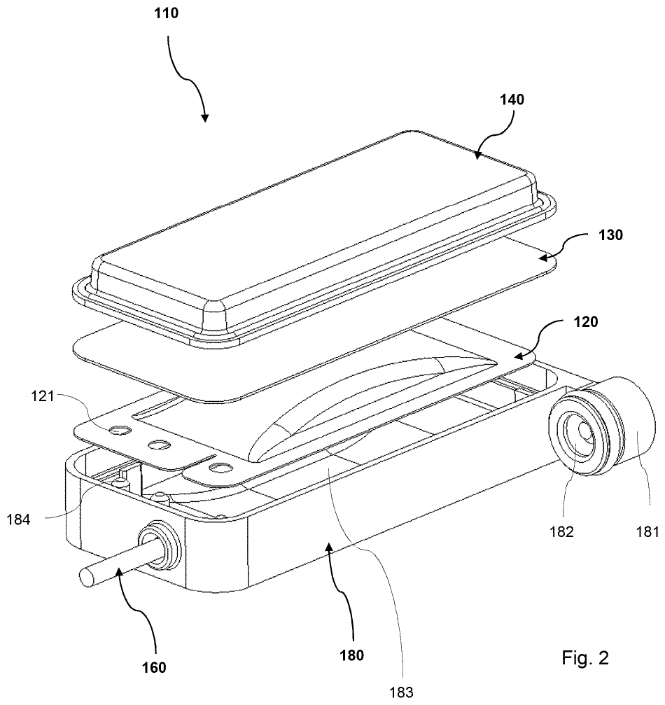

The term “osmotic actuator” 110 refers to osmotic actuators 110 with an osmotic membrane 130, preferably a flat sheet osmotic membrane, as shown in FIG. 2, but also to osmotic actuators 110 with two or more osmotic membranes 130.

The term “cavity with water” 140 refers to a solvent supply of the osmotic actuator 110, typically in the form of a flexible or collapsible reservoir, containing feed water.

Apart from such a cavity 140, the osmotic actuator 110 comprises a rigid pressure chamber 180, 280, 380 with one or more outlets 182, 282, 295, 395 and containing a draw solution. During use, the feed water passes through the osmotic membrane 130 from the cavity 140 to the pressure chamber 180, 280, 380.

The term “flat sheet membrane” refers to a semipermeable osmotic membrane 130 adapted to initiate an osmotic pressure in an osmotic actuator 110 by means of forward osmosis. The flat sheet membrane may be bended or shaped and it may also be in the form of a tubular membrane where this is considered advantageously.

The terms “feed water” and “solvent” refer to a solvent in the form of water or another kind of fluid with a lower salinity or a lower osmotic potential than the draw solution. The feed water is preferably in the form of demineralized water. It might also simply be referred to as “water” or “freshwater”.

The term “draw solution” refers to a solution containing an osmotic agent and with a higher salinity or osmotic potential than the feed water. Normally, the draw solution is made of a mix of water and a salt, e.g. NaCl₂, but sugar, polymers, alcohol, etc. mixed with water may also constitute a useful solution. Some clean liquids, such as different kind of alcohols, may also be used as draw solutions. The term “salt” is interchangeably used for any type of osmotic agent.

The term “drug-filled container” 102 refers to a compartment in form of a cartridge, a syringe or a flexible pouch containing a therapeutic agent.

The term “concentration polarisation” refers to the phenomenon that feed water from the cavity 140, which passes through the semi-permeable osmotic membrane 130 and into the osmotic actuator 110 containing the draw solution, accumulates near the osmotic membrane 130, whereby the osmotic potential falls. In other devices known in the art, it has been attempted to minimise the concentration polarisation by introducing certain spatial restrictions, which forces the flow through the pressure chamber only to move close to the osmotic membrane. The present invention, however, makes use of other solutions.

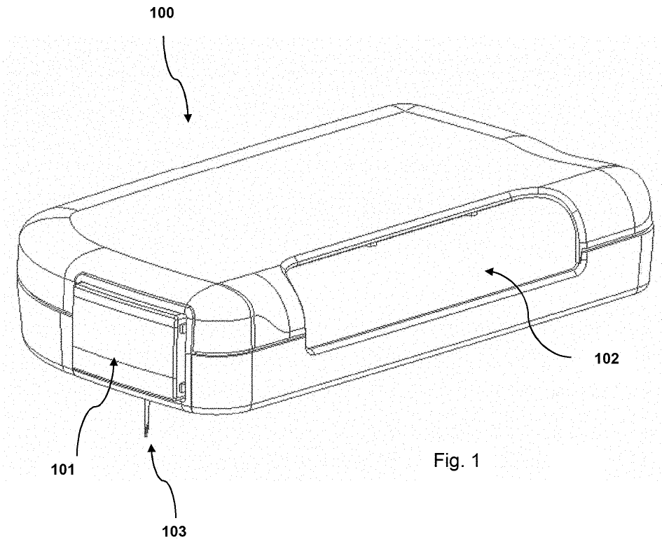

FIG. 1 shows a wearable injection device 100 comprising an osmotic actuator 110 (see FIG. 2). The illustrated injection device 100 is in its activated state, in which the push-button 101 for activating the injection device 101 has been pushed in, and the hypodermic needle 103 is in its extended position, where it is inserted into the skin of a patient. The drug-filled container 102 can be seen through an opening in the wearable injection device 100.

The functional sequence of the injection device 100 shown in FIG. 1 is as follows:
User peels off the protection paper (not shown) of the adhesive on the user interfacing side (not shown) of the injection device 100.
User attaches the injection device 100 to the body, e.g. in the abdominal region.
User pushes the push-button 101, which causes the hypodermic needle 103 to be inserted into the subcutaneous skin of the user and a flow path to be created between the drug-filled container 102 and the user.
During and immediately after the push of the push-button 101, the salt is released in the osmotic actuator 110. Feed water is drawn through an osmotic membrane 130 and the excess water/draw solution is lead to the drug-filled container 102.
A plunger in the drug-filled container 102 is moved by the excess water/draw solution, and the drug is pushed out through the hypodermic needle 103.
When the injection is fulfilled, the hypodermic needle 103 retracts automatically and a signal indicating that the injection is fulfilled is given to the user.
User removes the injection device 100 and disposes of it.

FIG. 2 shows an example of an osmotic actuator 110, in which different embodiments of flow rate stabilizing features may be implemented. As can be seen in the figure, the osmotic actuator 110 comprises a pressure chamber 180, which is connected via an adaptor 181 to an outlet 182 which, in turn, is meant to be connected with a drug-filled reservoir 102 (not shown in this figure). Inside the pressure chamber 180 is a cavity or compartment 183 comprising a pouch 120 with the draw solution and water surrounding the pouch 120.

A crushable glass ampoule 420 (see FIG. 11) filled with one or more salt tablets, with crystal salt or with an unsaturated, saturated or supersaturated salt solution may also be used as draw solution reservoir. The advantage of using glass as barrier is that glass has outstanding barrier properties to fluids and that a glass ampoule 420 can be easily broken by a crusher mechanism 461 integrated in the osmotic actuator 110 and actuated by the push-button 101. The filling end of the glass ampoule 420 may either be closed by melting the glass or with a plug or a seal. The advantage of using a plug or a kind of seal is that the filling percentage can be higher.

An osmotic membrane 130 and a cavity 140 with water are arranged on top of the pressure chamber 180, so that the osmotic membrane 130 constitutes a barrier between the pressure chamber 180 and the cavity 140 with water. The pouch 120 with the draw solution is positioned and fixed within the osmotic actuator 110 by means of holes 121 fitting over pins 184 in the pressure chamber 180.

A cutting device 160 is arranged to cut a hole in the pouch 120 with the draw solution to open it, when the injection is activated by pushing the push-button 101 (see FIG. 1), after which the pouch 120 is emptied, e.g. by means of elastomeric properties of the pouch 120 or by means of a spring (not shown), and the draw solution from the pouch 120 is mixed with the surrounding water in the pressure chamber 180. The release of the draw solution in the pressure chamber 180 can be carried out in many other ways and may be done by means of either a dry or a dissolved osmotic agent that is mixed with the surrounding water at activation of the injection device 100.

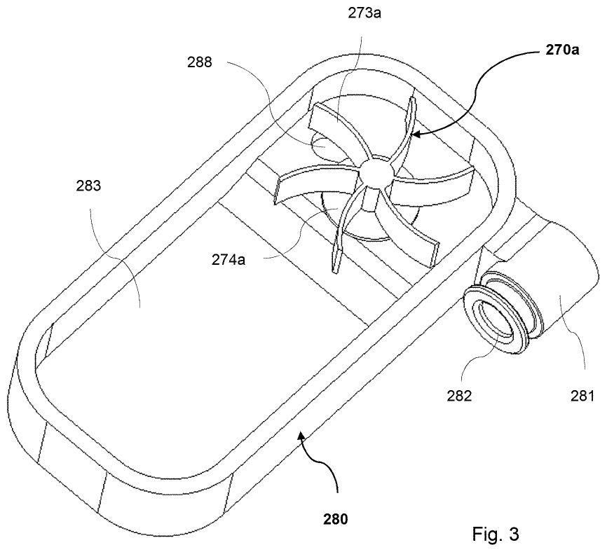

In FIGS. 3-5, an embodiment of the invention comprising a rotating element in the form of a centrifugal impeller 270a is illustrated. As shown in FIG. 3, the centrifugal impeller 270a is arranged in one end of the pressure chamber 280, while the draw solution pouch 120 and release mechanism (not shown) are positioned in the remaining compartment 283 of the pressure chamber 280.

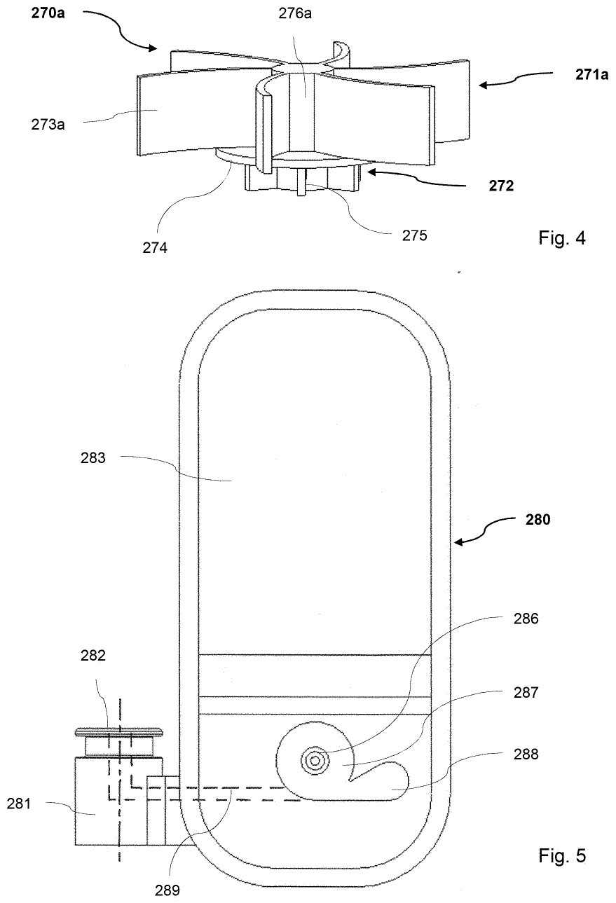

In FIG. 4, all elements of the centrifugal impeller 270a can be seen. The centrifugal impeller 270a has an upper part 271a comprising the impeller blades 273a for mixing the draw solution, and a lower part 272 with mill blades 275 rotated by the flow that is created when the feed water is pressed out of the pressure chamber 280, as will be explained below. A hollow centre part 276a is adapted to fit on a pin 286 in the pressure chamber 280 as shown in FIG. 5.

In FIG. 5, the pressure chamber 280 is shown the centrifugal impeller 270a. The lower part 272 of the centrifugal impeller 270a fits into a circular cut-out 287 in the pressure chamber 280, and a plate 274 on the centrifugal impeller 270a forms a roof to the cut-out 287. When feed water is drawn through the membrane 130, then the draw solution is pressed down in a droplet-shaped cut-out 288, which forms an inlet to the circular cut-out 287 which, in turn, is connected with an outlet channel 289 ending in the outlet 282.

The flow of draw solution through the circular cut-out 287 interacts with the mill blades 275 and forces the centrifugal impeller 270a to rotate. Thereby, the upper part 271a with the impeller blades 273a also rotates and circulation is created within the pressure chamber 280 and the draw solution is mixed with the incoming feed water.

It may be advantageously to fasten the centrifugal impeller 270a in its axial direction to ensure that the osmotic membrane 130 is not damaged during handling and use of the wearable injection device 100.

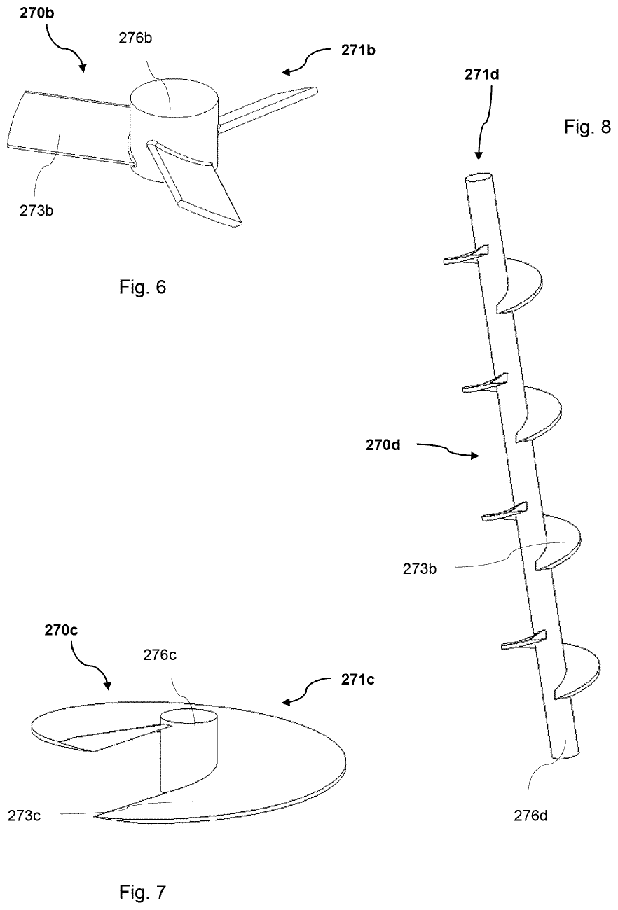

Other configurations of the rotating element than a centrifugal impeller can be used, some examples of which are shown in FIGS. 6-8. FIG. 6 shows a propeller 270b with propeller blades 273b arranged around its axial centre part 276b. Only the upper part 271b of the propeller 270b with the propeller blades 273b is shown, but a lower part 272 with mill blades 275 is also part of it (see FIG. 4 to for the lower part 272). Using a propeller 270b as rotating element creates an up and down stream and a circulation along the osmotic membrane 130 rather than a circulation along the walls of the pressure chamber 280, which is the case when using a centrifugal impeller 270a as illustrated in FIGS. 3-5.

In FIG. 7, the upper part 271c of a vertical helical spiral 270c with a blade forming one turn of an Archimedean spiral 273c on a vertical axis centre part 276c is illustrated. The vertical helical spiral 270c also creates and up and down stream but with a higher efficiency than the propeller 270b.

FIG. 8 shows an upper part 271d of horizontal helical spiral 270d where a number of one-turn Archimedean spiral blades 273d are arranged on a horizontal axis centre part 276d parallel to the osmotic membrane 130 of the osmotic actuator 110. This gives a better circulation than the other shown examples of rotation elements.

Both the vertical 270c and the horizontal 270d helical spirals shown in FIGS. 7 and 8, respectively, can be arranged with spiral blades that form more or less than one turn and both are equipped with a lower part 272 (not shown in these figures; see FIG. 4) to be driven to rotate and create circulation.

The main function of the above described rotation elements for mixing and circulation is to get the freshwater entering the pressure chamber 280 through the osmotic membrane 130 away from the osmotic membrane 130, so that draw solution with a higher salination, and osmotic potential can get in touch with the osmotic membrane 130. This can be done with simple circulation, which will mix the incoming water and the draw solution, or with more turbulent mixing as described above, but it can also be done by letting an impeller wheel as, for instance, the centrifugal impeller 270a shown in FIG. 4, touch the osmotic membrane 130 and scrape the incoming freshwater away. This is more efficient than simply circulating the liquids within the pressure chamber 280. The efficiency can be further increased by adding more impeller blades 273a or by angling the impeller blades 273a slightly to press the scraped water downward and away from the osmotic membrane 130.

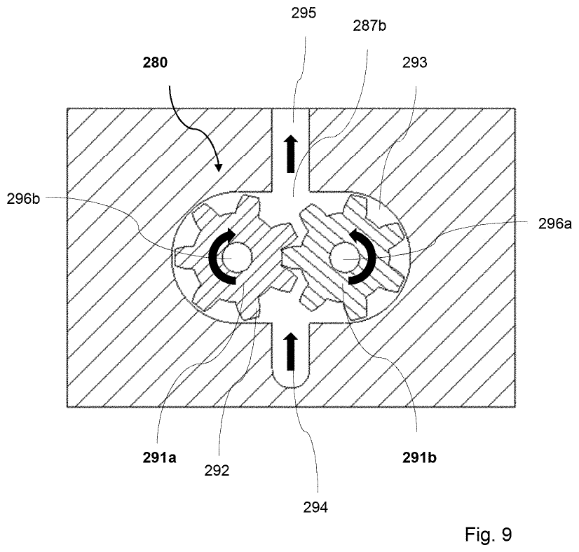

FIG. 9 shows a different way of driving the rotation elements by means of flow of draw solution. The lower part 272 of the rotation element is configured as a first gearwheel 291a, which cooperate with a second gearwheel 291b in a cut-out 287b in the pressure chamber 280.

When the water enters the pressure chamber 280 through the osmotic membrane 130, draw solution is forced through the inlet 294 and into the little cavities 293 formed by the gear wheel teeth 292 and the cut-out 287b in the pressure chamber. This forces the gear wheels 291a, 291b to rotate in opposite directions, and new cavities 293 move continuously from the inlet 294 to the gearwheels 291a, 291b towards the outlet 295 of the pressure chamber 280. Because the gear wheel teeth 292 of the two gear wheels 291a, 291b are in engagement with one another when moving from the outlet 295 towards the inlet 294, only a minimum of fluid is transferred in this “backwards” direction. A system like the described is called positive displacement and functions in the opposite way of a gear wheel pump because the gearwheels 291a, 291b have to move if a fluid is moving from the inlet 294 towards the outlet 295. This means that this system will be more efficient than the previously described system with mill blades 275. It is possible to use one or both of the gear wheel axes 296a, 296b for driving a rotation element 270a, 270b, 270c, 270d.

Other ways of forcing the rotation elements to rotate for mixing and circulation, which fall within the scope of the present invention can be imagined. Among these is an electric motor and different kinds of spring arrangements. In such cases, the lower part 272 of the rotation element 270a, 270b, 270c, 270d must be adapted to cooperate with these means.

A mechanism that, e.g., performs a repeated reciprocal motion back and forth rather than a rotational motion and fall within the scope of the present invention could also be envisioned.

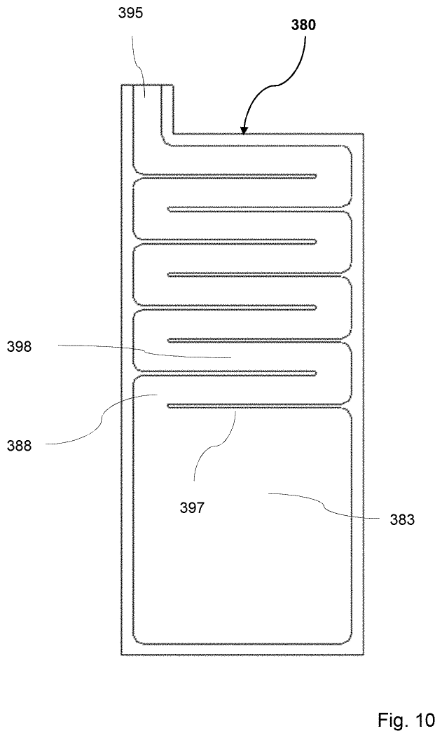

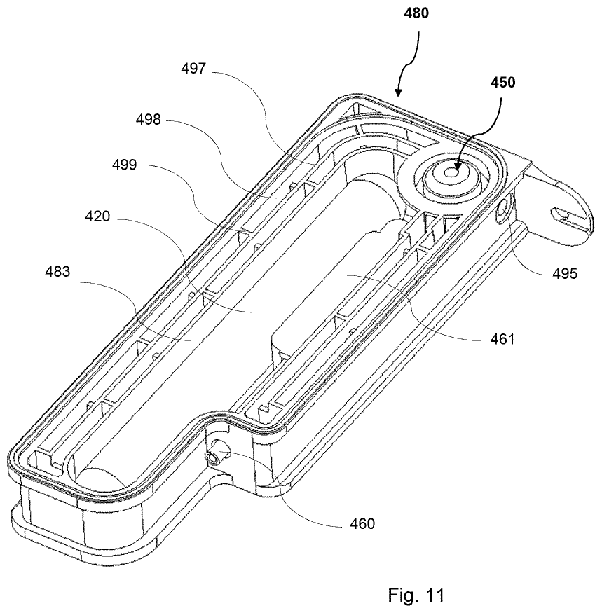

FIG. 10 shows an embodiment of the invention without any rotating elements. A pressure chamber 380 has a first compartment 383 for salt releasing and a second compartment 398 arranged as a long and twisted channel formed by the walls of the pressure chamber 380 and a number of internal walls 397. The second compartment 398 is positioned between the first compartment 383 and the outlet 395 of the pressure chamber 380.

When a salt release mechanism has released the salt at activation of the injection device 100 and a draw solution is created in the first compartment 383, the water entering through the osmotic membrane 130 (see FIG. 2) due to the osmotic potential in the first compartment, is pressed into the second compartment 398 formed by the twisted channel and water from the second compartment 398 is pressed out through the outlet 395. In the beginning, water will only be drawn into the pressure chamber in the region of the membrane over the first compartment 383. During the process more and more draw solution is pressed into the second compartment 398, causing the osmotic pressure over the second compartment 398 to increase. Therefore, more and more feed water is drawn through the osmotic membrane 130 in the region over the second compartment 398 as the injection progresses.

Because the liquid flow with a significant speed in the twisted channel of the second compartment 398, the draw solution and the incoming feed water are mixed to some degree and the efficiency is thereby increased. The channel in the second compartment 398 can be optimized by varying the length and width thereof and by adding different kind of obstructions in the channel to increase the fluid turbulence and mixing.

An advantage of this configuration is that the increasing effective area of the osmotic membrane 130 during the injection compensates for the diluted draw solution, so that the flow out of the outlet 395 to a drug-filled container 102 can be maintained constant.

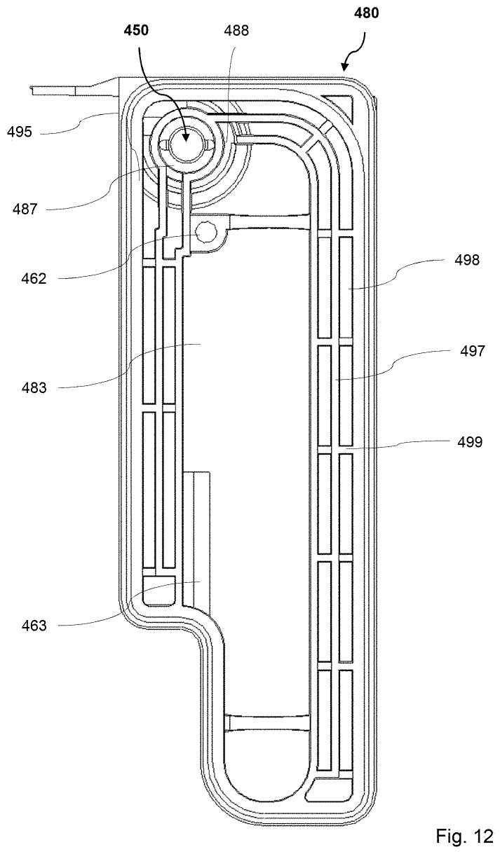

A disadvantage of the above configuration is a high sensitivity to the orientation of the osmotic actuator 110, especially if there is a large difference between the densities of the draw solution and of the feed water, respectively. In this case, the movement of the draw solution into the second compartment 398 will be speeded up in some orientations and slowed down in other orientations. FIGS. 11 and 12 show an alternative embodiment, which is less sensitive to orientation, as channels of the second compartment 498 surround the first compartment 483, in a way so that the fluid has to move both up and down and to both sides before reaching the outlet 495, independently of the orientation of the osmotic actuator 110.

In FIG. 11 the pressure chamber 480 is shown without the membranes in the top and bottom so that the interior of the pressure chamber 480 is visible. The first compartment 483 contains a crushable glass ampoule 420 surrounded by water and filled with one or more salt tablets, with crystal salt or with an unsaturated, saturated or super saturated salt solution. In cases, in which the glass ampoule 420 is filled with salt tablets or crystal salt, vacuum may be applied to the glass ampoule 420 before it is sealed to minimise the amount of air within the glass ampoule 420.

A crusher pin 460, which is moved through a distance when the push-button (not shown) is pushed, is arranged to interact with a crusher mechanism 461 to crush the glass ampoule 420. In the shown embodiment, the crusher mechanism 461 is arranged to be rotated into the glass ampoule 420 about an axis perpendicular to the membranes (not shown) and placed in the end of the crusher mechanism 461 facing away from the crusher pin 460, but many other ways of crushing the glass ampoule 420 may be envisioned.

When the glass ampoule 420 has been crushed, the crystal salt will dissolve or the salt solution will mix with the surrounding water within the pressure chamber 480, and an osmotic pressure will build up. This will cause feed water to be drawn in from feed water reservoirs on the other side of the membranes, which feed water reservoirs may be flexible or rigid and connected with, e.g., one common or two separate flexible pouches (not shown). Hereafter, the excess water and draw solution will move into the second compartment 498, which is formed by longitudinal walls 497 and the upper and lower membranes (not shown). Transversal walls 499, which alternately open for flow in the top and in the bottom, are spread out in the channel between the longitudinal walls 497 on a regular basis to obstruct and mix the flow, and to cause the draw solution to be in contact with as much of the membrane area as possible.

Before reaching the outlet 495, the water/draw solution will pass through an outlet compartment 487 (see FIG. 12), in which a relief valve 450 is arranged. The function of the relief valve 450 is to by-pass the water/draw solution back into the feed water compartments if the outlet 495 is blocked, e.g. when the plunger in the drug-filled container 102 (see FIG. 1) has reached the end position or is blocked by error.

In FIG. 12, the flow pathway can be seen more clearly. When a draw solution has been provided in the first compartment 483 and water is drawn into the first compartment 483, the excess water/draw solution is pressed through the entrance 488 and into the second compartment 498. Hereafter, the water/draw solution will first move down and then back up in the channels to the right, then move across in the channel in the top to the channels at the left, and then again move down and back up until the outlet compartment 487 with the relief valve 450 is reached. From here, it moves to the outlet 495 and into the drug-filled container 102 to push the plunger in the drug-filled container 102 and expel the drug. Also visible in FIG. 12 is the pivot 462 for the crusher mechanism 461 and the inlet channel 463 for the crusher pin 460.

Other kinds of mixing arrangements falling within the scope of the invention may be envisioned. These can, for instance, be based on the following principles:

### Electricity

Because the draw solution contains ions, electricity can be produced by placing an anode of, e.g., zinc in one end and a cathode of, e.g., carbon or cobber in the other end of the pressure chamber containing the draw solution and connecting the anode and the cathode to each other through an electrical resistance. If, for instance, a grid made of zinc forming the anode is placed right under the osmotic membrane 130 and a plate made of carbon or cobber forming the cathode is placed in the opposite side of the pressure chamber, more or less all the draw solution is between the anode and cathode. Then the negative ions in the draw solution are drawn against the zinc anode and the positive ions are drawn against the cathode, and a high number of ions near the osmotic membrane 130 is obtained. The electrical resistance may be in the form of an electric light bulb, an LCD display or the like, which could be used for giving information to the user regarding the operational status of the osmotic actuator 110 and the injection device 100.

### Magnetism

Diamagnetism refers to an object's tendency to generate a weak magnetic field in opposition to a magnetic field applied to it. Diamagnetic objects repel magnets, and because water is diamagnetic, it repels magnets and tends to move in a direction away from an external magnet. However, salt reduces the diamagnetic properties of water and salt water does not repel an external magnetic field as much as freshwater. A mixing effect will therefore be created when a strong magnet is positioned near a non-homogenous blend of water and salt water, as the freshwater will be repelled into and mixed with the salt water.

An alternative way of making use of an external magnet is by adding a ferromagnetic and granulated material, e.g. in the form of magnetite (Fe₃O₄) nanoparticles, to the draw solution. As these particles have a surface charge, they will attract layers of negative and positive ions onto their surfaces. If a magnet is placed outside the osmotic membrane 130, either in the feed water cavity 140 or outside the feed water cavity 140, the magnetic field will attract the nanoparticles (and the ions present on their surfaces) and make them move towards the osmotic membrane 130, resulting in a higher density of ions near the osmotic membrane 130.

### Hydraulic Vortices or Swirls

This principle is known from vortex flow meters, which measure fluid velocity using a principle of operation referred to as the von Kármán effect. It states that, when a flow passes by a bluff body, a repeating pattern of swirling vortices is generated. An obstruction in the flow path causes fluid to separate and form areas of alternating differential pressure known as vortices around the backside of the bluff body. The result is that the fluid is being swirled around and mixed behind the bluff body.

### Chemistry

By adding wetting agents to a non-homogenous salt solution to lower the internal resistance in the solution, it is possible to increase the mixing speed by diffusion. Another solution is to add one or more chemicals acting as catalysts in a way that increases the mixing speed of a non-homogenous salt solution. Finally, chemicals that produce a gas, e.g. CO₂, when it comes into contact with certain osmotic agents or with water, and which thereby will create some internal movement in the solution, could be added.

### Multiple Outlets

Another area of solutions implies that the outlet flow from the pressure chamber is switching between two or more outlets. An object in the pressure chamber is moved between the outlets due to changing pressure conditions near the outlets, and this object is, for instance, capable of operating a preferably bi-stable mechanism between two positions, which in turn opens and closes two outlets, respectively.

### Flexible Pressure Chamber

A pressure chamber that is capable of expanding slightly with increasing pressure in combination with a flow restrictor in the outlet will provide a more stable outlet flow. In the beginning, when the salt concentration in the pressure chamber is high and no concentration polarisation is present, a high pressure is generated within the pressure chamber. However, if the flow restrictor provides an increasing resistance with increasing flow, a balance between flow, pressure and expansion will arise. During the injection, when the pressure decreases due to concentration polarisation and dilution of the draw solution, the resistance through the flow restrictor also decreases, and a new balance, with a lower (or no) expansion of the pressure chamber, arises. Thereby, the expanded volume in the beginning of the injection has been delivered at a later time, and a more stable flow over time has been provided.

Slow Release of Salt Water from the Pouch

The salt water from the salt water pouch 120 can be released slowly by making a hole of a small and well-controlled size therein at activation of the injection device 100. If a spring force acting on the pouch 120 is known and controllable, it is possible to control and prolong the time for emptying the pouch 120 so that a more constant salt concentration within the draw solution throughout the injection is maintained.

### LIST OF REFERENCE NUMBERS

100. Injection device
101. Push-button for activating injection device
102. Drug-filled container
103. Hypodermic needle
110. Osmotic actuator
120. Pouch with draw solution
121. Hole for positioning and fitting pouch
130. Osmotic membrane
140. Cavity containing water
160. Cutting device
180. Pressure chamber
181. Adaptor between pressure chamber and outlet
182. Outlet from pressure chamber
183. Compartment in pressure chamber
184. Pin for positioning and fitting pouch
270a. Centrifugal impeller
270b. Propeller
270c. Vertical helical spiral
270d. Horizontal helical spiral
271a. Upper part of centrifugal impeller
271b. Upper part of propeller
271c. Upper part of vertical helical spiral
271d. Upper part of horizontal helical spiral
272. Lower part of rotating element
273a. Impeller blade
273b. Propeller blade
273c. One-turn Archimedean spiral blade
273d. One-turn Archimedean spiral blade
274. Plate on rotating element
275. Mill blade of rotating element
276a. Centre part of centrifugal impeller
276b. Centre part of propeller
276c. Centre part of vertical helical spiral
280. Pressure chamber
283. Compartment in pressure chamber
282. Outlet from pressure chamber
286. Pin in pressure chamber for centrifugal impeller
287. Circular cut-out in pressure chamber
287b. Cut-out in pressure chamber for gear wheels
288. Droplet-shaped cut-out in pressure chamber
289. Outlet channel in pressure chamber
291a. First gear wheel
291b. Second gear wheel
292. Gear wheel tooth
293. Cavity between gear wheel teeth
294. Inlet to gear wheels
295. Outlet from pressure chamber
296a. First gear wheel axis
296b. Second gear wheel axis
380. Pressure chamber
383. First compartment of pressure chamber
395. Outlet from pressure chamber
398. Second compartment of pressure chamber
420. Glass ampoule
450. Relief valve
460. Crusher pin
461. Crusher mechanism
462. Pivot for crusher mechanism
463. Inlet channel for crusher pin
480. Pressure chamber
483. First compartment of pressure chamber
487. Outlet compartment of pressure chamber
488. Entrance to second compartment
495. Outlet from pressure chamber
497. Longitudinal walls of second compartment
498. Second compartment of pressure chamber
499. Transversal walls of second compartment

1. An osmotic actuator for an injection device adapted for subcutaneous injection of a medicament into the tissue of a user, which osmotic actuator comprises
a pressure chamber having one or more outlets and containing a draw solution,
one or more osmotic membranes,
a cavity containing a solvent, and
a dilution-compensating arrangement,
wherein the one or more osmotic membranes forms a part of an internal surface area of the pressure chamber, and wherein the cavity containing the solvent abuts at least part of an external surface of the one or more osmotic membranes, so that at least one common boundary between the pressure chamber and the cavity containing the solvent is formed by the one or more osmotic membranes, and
wherein the dilution-compensating arrangement is arranged to compensate for the dilution of the draw solution near the one or more osmotic membranes, which dilution occurs when solvent from the cavity enters the pressure chamber through the one or more osmotic membranes.

2-3. (canceled)

4. The osmotic actuator according to claim 1, wherein the dilution-compensating arrangement comprises a rotating element defining an axis of revolution and with an upper part having one or more protrusions spaced equally around the axis of revolution,
the rotating element being positioned in the pressure chamber and arranged to rotate at least during a part of the time, in which the osmotic actuator is active.

5. The osmotic actuator according to claim 4, wherein the rotating element has a lower part with three or more protrusions spaced around the axis of revolution, the lower part being arranged in an outlet flow of the pressure chamber in such a way that, when the draw solution flows towards the one or more outlets of the pressure chamber, the flow causes the rotating element to rotate around the axis of revolution.

6-9. (canceled)

10. The osmotic actuator according to claim 1, wherein the draw solution contains magnetic particles having a positive or negative electrical surface charge.

11. The osmotic actuator according to claim 1, wherein the dilution-compensating arrangement comprises an anode and a cathode being connected through an electric resistance and positioned within the pressure chamber,
wherein one of the anode and the cathode is positioned near the one or more osmotic membranes and the other is placed in an opposite side of the pressure chamber, so that the main part of the draw solution is positioned between the anode and the cathode.

12-14. (canceled)

15. The osmotic actuator according to claim 1, wherein the dilution-compensating arrangement comprises a division of the pressure chamber into a first compartment, into which the draw solution is released at activation of the injection device, and a second compartment arranged between the outlet and the first compartment, the second compartment being configured to be longer and narrower than the first compartment.

16. The osmotic actuator according to claim 15, wherein one or more protrusions are arranged in the second compartment to partly obstruct the flow through the second compartment.

17-18. (canceled)

19. The osmotic actuator according to claim 1, wherein the dilution-compensating arrangement comprises a bluff body to be passed by incoming solvent in the osmotic actuator for creation of hydraulic vortices.

20. The osmotic actuator according to claim 1, wherein the dilution-compensating arrangement comprises chemicals added to the draw solution for increasing the speed of mixing by diffusion and/or for creating some movement in the draw solution.

21. The osmotic actuator according to claim 1, wherein the dilution-compensating arrangement comprises a body, which is arranged within the osmotic actuator and to repeatedly shift position, thereby defining which one of a plurality of outlets is open at any given time, the other outlets being closed.

22. The osmotic actuator according to claim 1, wherein the dilution-compensating arrangement comprises an expandable pressure chamber combined with a flow restrictor, which only allows a certain amount of fluid to pass through the one or more outlets.

23. The osmotic actuator according to claim 1, wherein the draw solution within the pressure chamber is obtained by breaking a watertight barrier of a watertight salt depository arranged within the pressure chamber during activation of the osmotic actuator, whereby one or more salt tablets, crystal salt or an unsaturated, saturated or supersaturated salt solution initially contained by the watertight salt depository is brought into contact with water surrounding the watertight salt depository within the pressure chamber.

24. The osmotic actuator according to claim 23, wherein the watertight salt depository is a glass ampoule.

25. (canceled)

26. The osmotic actuator according to claim 23, wherein the breaking of the watertight barrier is fully or partly caused by a physical force applied to the watertight salt depository.

27. (canceled)

28. The osmotic actuator according to claim 23, wherein the porosity of salt tablets or crystal salt initially contained by the watertight salt depository is reduced by stamping the salt crystals of the crystal salt before enclosing the crystal salt by the watertight barrier.

29-31. (canceled)

32. The osmotic actuator according to claim 23, wherein the initial amount of water surrounding the watertight salt depository within the pressure chamber is insufficient to dissolve the total amount of salt tablets or crystal salt initially contained by the watertight salt depository.

33. The osmotic actuator according to claim 1, wherein the one or more osmotic membranes are flat sheet membranes.

34. An injection device comprising an osmotic actuator according to claim 1.

35. The injection device according to claim 34, wherein the injection device and the osmotic actuator are arranged so that the amount of medicament injected during the use of the injection device is within the range from 1 ml to 20 ml.
